(19) United States
(12) Patent Application Publication
(10) Pub. No.: US 2025/0152490 A1
(43) Pub. Date: May 15, 2025

(54) COMPOSITIONS COMPRISING PIROCTONE OLAMINE

(71) Applicant: CLARIANT INTERNATIONAL LTD., Muttenz (CH)

(72) Inventors: Dongzhi CHEN, Shanghai (CN); Chenjiang ZHU, Shanghai (CN); Xiang WANG, Shanghai (CN); Mingli XU, Shanghai (CN); Jinglan LI, Shanghai (CN)

(21) Appl. No.: 18/839,496

(22) PCT Filed: Mar. 4, 2022
(86) PCT No.: PCT/CN2022/079330

§ 371 Date: Aug. 19, 2024

(51) Int. Cl. A61K 8/49; A61Q 5/00; A61Q 5/02

(52) U.S. Cl. CPC A61K 8/4926; A61Q 5/006; A61Q 5/02

(57) ABSTRACT

The present invention provides a composition comprising the following components: 0.1-15 wt.-% of piroctone olamine, 17.5-28 wt.-% of surfactant, 15-35 wt.-% of alcohol solvent, and 0.1-2 wt.-% of fatty acid ester or oil, wherein the weight percentage of each component is based on the total weight of the composition. The invention also provides a method for preparing the composition and relates to uses of the composition in personal or home care products.

### TECHNICAL FIELD

The present invention relates to compositions comprising piroctone olamine and to the use of the compositions in personal care or home care products.

### BACKGROUND

With fast development of production and economy, the demand for care products, especially personal care products such as shampoo or conditioner, is increasing.

People want their hair to be clean, smooth, beautiful and elastic after using a quality shampoo or hair care products. However, in recent years, dandruff has become a problem for many consumers.

Researchers found that abnormal growth of Malassezia is an important cause of dandruff. Conventional treatments for dandruff include the use of anti-dandruff agents such as zinc pyrithione present in the medium, particularly in shampoos, gels or lotions, in order to disperse the anti-dandruff agent and enable it to be deposited on the skin. The disadvantage of using pyrithione salt formulations has been found to be the adverse effect on keratin fibers, especially sensitized keratin fibers, which reduces the aesthetic properties of the hair, in particular by making hair rougher and more charged.

Researchers developed piroctone olamine. Piroctone olamine is an antifungal active agent. German patent applications DE2234009A1 and DE1795270A1 disclose the formula of piroctone olamine. German patent application DE1795270A1 also describes a method for preparing piroctone olamine. Piroctone olamine exists in crystalline form and can be added to personal care products to effectively reduce or eliminate dandruff. In addition, patent application WO2002/08196 describes the use of piroctone olamine as preservative.

The demand of high-quality anti-dandruff product is increasing. Consumers are looking for a multi-functional anti-dandruff product with excellent solubility and wide application. Therefore, a new composition comprising piroctone olamine with a good balance between effective anti-dandruff and preservative effects with excellent solubility is desired.

### SUMMARY OF THE INVENTION

In a first aspect, the present invention provides a composition comprising the following components:
A) 0.1-15 wt.-% of piroctone olamine,
B) 17.5-28 wt.-% surfactant.
C) 15-35 wt.-% of alcohol solvent, and
D) 0.1-2 wt.-% of fatty acid ester or oil,
the weight percentage of each component being based on the total weight of the composition.

In a second aspect, the present invention also provides personal care products or home care products comprising 0.2-10 wt.-% of the above-mentioned composition in addition to 8-30 wt.-% of a further surfactant, conditioning agent of 0-10 wt.-%, and water.

In a third aspect, the present invention also provides a method for producing the above composition at room temperature, which comprises adding 0.1-15 wt.-% of piroctone olamine to 15-35 wt.-% of alcohol solvent, stirring to dissolve it completely, mixing the above dissolved substance with 17.5-28 wt.-% of surfactant and stirring further, and then adding 0.1-2 wt.-% of fatty acid ester or oil and 25-55 wt.-% of water to dissolve, the weight percentage of each component is based on the total weight of the above composition.

### DESCRIPTION OF THE INVENTION

For the avoidance of doubt, any feature of an aspect of the invention may be used in any other aspect of the invention. The term “comprising” is intended to mean “including” but does not necessarily mean “consisting of” or “composed of”. In other words, the list of steps or options need not be exhaustive. Note that the embodiments given in the following specification are intended to illustrate the invention and are not intended to limit the invention to those embodiments per se. Similarly, all percentages are weight/weight percentages unless otherwise stated. When multiple preferred ranges are described in the form of “x to y” for a particular feature, it is understood that all ranges at different endpoints of the combination are also considered. The various features of the invention mentioned in each of the above sections apply, mutatis mutandis, to the other sections as appropriate. Thus, features specified in one section may be appropriately combined with features specified in other sections. The addition of any section headings is for convenience only and is not intended to limit the present disclosure in any way.

The term “cold process” means that the various materials or components are dissolved and mixed completely without heating.

The present invention provides a composition, particularly for use in personal care or home care products. The composition of the present invention comprises the following components: 0.1-15 wt.-% of piroctone olamine, 17.5-28 wt.-% of surfactant, 15-35 wt.-% of alcohol solvent, 0.1-2 wt.-% of fatty acid ester or oil, the weight percentage of each component being based on the total weight of the composition. The present invention also provides the application of above-mentioned composition in the preparation of personal care product or home care product.

Each component of the invented composition and application in personal care products such as shampoos are explained as embodiments and described in more details below.

### Component A

The composition of the present invention comprises component A, i.e., piroctone olamine. As mentioned earlier, because of the antifungal properties of the piroctone olamine, it can inhibit the fungus that causes dandruff such as the common Malassezia.

As an exemplary embodiment, the weight percentage of piroctone olamine is 0.1-15%, preferably 0.5-15%, more preferably 1-15%, more preferably 2-15%, more preferably 4-15%, more preferably 6-12%, more preferably 8-12%, most preferably 8-10%, based on the total weight of the composition.

### Component B

The composition of the present invention comprises component B, i.e. surfactants. As used herein, “surfactant” means surface active agent. The surfactant may be an anionic surfactant, an amphoteric surfactant, a cationic surfactant, a nonionic surfactant, or mixtures thereof. In at least one embodiment, the surfactant is an organic amphiphilic compound having at least one hydrophobic portion and at least one hydrophilic portion.

In the present invention, the weight percentage of surfactant is 17.5-28%, more preferably 19-25%, most preferably 20-23%, based on the total weight of the composition. Within this range, piroctone olamine has good solubility when dispersed in the surfactant. In the present invention, the surfactant is used as the main solvent for piroctone olamine, and therefore the surfactant accounts for a relatively large percentage of the composition of the present invention.

In another exemplary embodiment, the surfactant component B is an anionic surfactant. Ammonium lauryl ether sulfate and sodium lauryl ether sulfate (SLES) are particularly preferred surfactants for use in hair care compositions of this invention. Specific examples of other suitable anionic surfactants include polyoxyethylene alkyl ether sulfates such as sodium laureth sulfate, isethionate, taurate, sodium C₁₄₋₁₆ olefin sulfonate, ammonium C₁₂₋₁₅ pareth sulfate, sodium myristyl ether sulfate, or polyoxyethylene alkyl sulfates such as trialcoholamine lauryl sulfate, sodium lauryl sulfate, disodium monooleamidosulfosuccinate, ammonium lauryl sulfosuccinate, sodium dodecylbenzene sulfonate, trialcoholamine dodecylbenzene sulfonate, and sodium N-lauroyl sarcosinate.

In another exemplary embodiment, the surfactant component B is a non-ionic surfactant, which may be selected from alkoxylated derivatives of the following: fatty alcohols, alkyl phenols, fatty acids, fatty acid esters and fatty acid amides, in the C₁₆ to C₄₀ range, and having from about 1 to about 110 alkoxy groups. The alkoxy groups are selected from the group consisting of C₂₋₆ oxides and their mixtures, with ethylene oxide, propylene oxide, and their mixtures being the typical alkoxides. The alkyl chain may be linear, branched, saturated, or unsaturated. Of these alkoxylated nonionic surfactants, the ethoxylated alcohols and propoxylated alcohols are preferred. The alkoxylated alcohols may be used alone or in mixtures.

Other nonionic surfactants suitable for use in the present invention are alkyl glycosides, which are the condensation products of long chain alcohols, e.g. C₈₋₃₀ alcohols, with sugar or starch polymers. Commercially available examples of these surfactants include decyl glucoside and lauryl polyglucoside. Also suitable are glucamide surfactants, for example those under the brand name of Gluco Tain® or GlucoPure® (Clariant). Also useful herein as nonionic surfactants are sorbitan esters such as sorbitan monopalmitate. Sorbitan esters may comprise mixtures of mono-, di-, tri-esters. Representative examples of suitable sorbitan esters include sorbitan monooleate, sorbitan stearates, sorbitan monoisostearate, and sorbitan sesquioleate.

In another exemplary embodiment, nonionic surfactants are glyceryl esters and polyglyceryl esters, including glyceryl monesters, typically glyceryl monesters of C₁₆₋₂₂ saturated, unsaturated, linear and branched chain fatty acids such as glyceryl oleate, glyceryl monostearate, glyceryl monoisostearate, glyceryl monopalmitate, glyceryl monobehenate, and mixtures thereof.

Also useful herein as nonionic surfactant may be selected from the group comprising fatty acid dialcoholamides (DEA). Typical examples are isostearic acid DEA, lauric acid DEA, capric acid DEA, linoleic acid DEA, myristic acid DEA, oleic acid DEA, and stearic acid DEA.

Further nonionic surfactants are selected from the group comprising fatty acid monoalcoholamides such as coconut fatty acid monoalcoholamide, fatty acid monisopropanolamides such as oleic acid monoisopropanolamide, lauric acid monoisopropanolamide, alkyl amine oxides such as N-cocodimethylamine oxide, N-lauryl dimethylamine oxide, N-myristyl dimethylamine oxide, and N-stearyl dimethylamine oxide, N-acyl amine oxides such as N-cocoamidopropyl dimethylamine oxide and N-tallowamidopropyl dimethylamine oxide, and N-alkoxyalkyl amine oxides such as bis(2-hydroxyethyl) C₁₂₋₁₅ alkoxy-propylamine oxide.

In another preferred embodiment, the surfactant component B is an amphoteric surfactant, which can act as anionic surfactant in an alkaline solution or as cationic surfactant in an acidic solution. Examples are cocoamphocarboxyglycinate, cocoamphocarboxypropionate, cocobetaine, cocamidopropyl betaine, N-cocamidopropyldimethylglycine, N-lauryl-N-carboxymethyl-N-(2-hydroxyethyl)ethylenediamine.

Betaines such as alpha-(tetradecyldimethylammonio)acetate, beta-(hexadecyldiethylammonio) propionate, and gamma-(dodecyldimethylammonio) butyrate and sultaines such as 3-(dodecyldimethylammonio)-propane-1-sulfonate, and 3-(tetradecyldimethylammonio) ethane-1-sulfonate are named. The above-mentioned surfactants can be used alone or in combination in the compositions according to this invention.

### Component C

The composition of the present invention comprises component C, i.e., alcohol solvent. In an exemplary embodiment, the weight percentage of alcohol solvent is 15-35%, preferably 16-30%, more preferably 17-25%, based on the total weight of the composition of the present invention. Within the above selection range, piroctone olamine has better solubility and also enhances the antibacterial properties of the composition.

Suitable alcohol solvent includes aqueous solutions of lower alkyl alcohols or polyols. The lower alkyl alcohols may be, for example, monoalcohols having 1-6 carbon atoms, typically ethanol and/or isopropanol. Polyols may be, for example, propylene glycol, hexylene glycol, glycerol and/or propylene glycol. As an illustrative example, an alcohol solvent suitable for the present invention is selected from propylene glycol, butylene glycol, pentylene glycol, ethanol hexanediol and mixtures thereof.

### Component D

The composition of the present invention comprises component D, i.e., fatty acid ester or oil. The fatty acid ester or oil in the composition of the present invention may enhance the antibacterial properties of the composition. In an exemplary embodiment, the weight percentage of the fatty acid ester or oil is 0.1-2%, preferably 0.2-1.5%, more preferably 0.5-1.0%, based on the total weight of the composition of the present invention.

Suitable oil of the present invention may include vegetable oils such as castor oil, coconut oil, lentil oil, apricot kernel oil, avocado oil, babassu oil, evening primrose oil, linseed oil, grape seed oil, macadamia seed oil, corn oil, white manzanita seed oil, olive oil, palm kernel oil, safflower oil, sesame oil, soybean oil, sunflower oil, wheat germ oil and Dianthus seed oil.

Other suitable fatty acid ester includes but not limited to anhydrous sorbitol derivatives, sorbitan caprylate, sorbitan caprate, sorbitan oleate, sorbitan stearate. Other suitable oils and fats are, for example, PEG anhydrous sorbitan beeswax, PEG anhydrous sorbitan isostearate, PEG anhydrous sorbitan lanolate, PEG anhydrous sorbitan laurate, PEG anhydrous sorbitan oleate, PEG anhydrous sorbitan palmitate, PEG anhydrous sorbitan stearate, polysorbate, anhydrous sorbitan trioleate, anhydrous sorbitan sesquioleate, anhydrous sorbitan stearate, and sorbitan tristearate. Other suitable fatty acid ester include, but are not limited to, sorbitan fatty acid esters, sorbitan caprylate, glycerol esters of fatty acids, or triglycerides.

The compositions of the present invention can be added to personal care products or home care products. Examples of personal care products include but are not limited to shampoos, conditioners, hair regrowth agents, hand soaps, face washes, body washes, cleansing masks, cleansing lotions, micellar waters, makeup removers, cleansing wipes, fragrances, soaps, shaving soaps, shaving foams, cleansing foams, face masks, creams and hand creams for the care of personal hair, skin and other body parts. Examples of home care products include, but are not limited to, laundry care products, air fresheners and home cleaners.

Preferably, the composition of the present invention is added or applied to a hair care composition, a scalp care composition or a skin care composition. More preferably, the compositions of the present invention are applied or added to shampoo compositions to have anti-dandruff and preservative properties.

In an exemplary embodiment, mix 0.2-10 wt.-% of the composition of the present invention, 8-30 wt.-% of a second or further surfactant with 0-10 wt.-% of a conditioning agent and water to make an anti-dandruff and antiseptic shampoo composition. In an exemplary embodiment, the weight percentage of the further surfactant is 10-30 wt.-%, preferably 12-20 wt.-%, most preferably 15-20 wt.-%. As an exemplary embodiment, the further surfactant comprises at least one of an anionic surfactant, a nonionic surfactant, and an amphoteric surfactant. In a preferred embodiment, the further surfactant comprises any combination of at least one or more of alkylamidopropyl betaine, alkyl betaine, sodium alkyl acyl sarcosinate, sodium dodecyl polyether sulfate.

The personal care product or home care product of the present invention may also include water, preferably deionized water.

### Component F

In another exemplary embodiment, the personal care product or home care product of the present invention further comprises one or more additional component(s) (F) in an amount of at least 0.01 wt.-%, preferably at least 0.05 wt.-%, more preferably at least 0.1 wt.-%, and even more preferably at least 0.5 wt.-% of the total weight of the personal care or home care composition/product.

Preferably, component (F) is selected from acidity regulators, coloring agents, conditioning agents, emulsifiers, film-forming agents, fragrances, glossing agents, wetting agents, lubricants, humectants, pigments, preservatives, skin penetration enhancers, stabilizers, thickeners and viscosity modifiers. More preferably, component (F) is selected from acidity regulator, glossing agent, lubricant. Suitable lubricants are, for example, fatty alcohol components with 6-18 carbon atoms.

In at least one embodiment, component (F) is a thickener. In a preferred embodiment, the thickener comprises at least one or more of hydroxyethyl cellulose, hydroxypropyl cellulose, carboxymethyl cellulose, guar gum, xanthan gum, acrylic (ester) copolymer or acrylamidomethylpropanesulphonic acid homo- or co-polymer (e.g. commercially available as Aristoflex® from Clariant).

In at least one embodiment, component (F) is a glossing agent. Typical glossing agents are siloxanes. Suitable for use as siloxanes are volatile or non-volatile non-ionic silicone fluids, silicone resins and silicone semi-solids or solids. Volatile siloxanes are linear or cyclic siloxanes with a measurable vapor pressure, defined as a vapor pressure of at least 2 mm Hg at 20° C. Also suitable are water-insoluble non-volatile silicone fluids, including polyalkylsiloxanes, polyaryl siloxanes, polyalkylaryl siloxanes, polyether siloxane copolymers, amine-functional siloxanes, or mixtures thereof.

In at least one embodiment, component (F) is a rheology modifier. The weight percentage of the rheology modifier is 0.1-10%, preferably 0.2-5%, more preferably 0.2-3%, also more preferably 0.5-5%, based on the total weight of the personal care product, such as anti-dandruff shampoo. Rheology modifiers can be gelling or thickening agents. Examples of thickeners include, but are not limited to, cellulose thickeners such as hydroxyethyl cellulose, hydroxypropyl cellulose and carboxymethyl cellulose, guar gums such as hydroxypropyl guar gum, gums of microbial origin such as xanthan gum and dextran gum, and synthetic thickeners such as cross-linked homopolymers or copolymers of acrylic acid and/or acrylamide-based propionic acid, or acrylamidomethylpropanesulphonic acid homo- or co-polymers (e.g. commercially available as Aristoflex® from Clariant). Other rheology modifiers include fatty acid amides such as coconut diethanolamide and monoethanolamide, and oxyethyleneated monoethanolamides of carboxylic acid alkyl ethers.

In at least one embodiment, component (F) is a conditioning agent, and in an exemplary embodiment, the amount of the conditioning agent is 0-10%, preferably 0.5-10%, more preferably 2-8% by weight, based on the total weight percentage of the composition of the invention. Examples of suitable conditioning agents include cationic quaternary ammonium salts. In at least one embodiment, the component (F) is a cationic quaternary ammonium salt. Examples of such quaternary ammonium salts include benzyl triethyl ammonium chloride, cetyl trimethylammonium chloride (cetrimonium chloride, CTAC), behentrimonium chloride (BTAC) or cetylpyridinium chloride. Further suitable conditioning agents include Quaternium-98 (Genadvance® Repair), Polyquaternium-116 and Butylene Glycol (Genadvance® Life), Lauryl/Myristyl Polyricinoleate and Glycerin (Genadvance® Hydra), Stearamidopropyl Dimethylamine (Genamin® SPA) or distearyl dimethyl ammonium chloride (Genamin® DSAP).

Examples of further components (F) are panthenol, panthenyl ethyl ether, proteins, hydrolyzed proteins (preferably of plant or animal origin, such as hydrolyzed collagen or hydrolyzed keratin), nutrients; antioxidants, such as vitamin E; emollients, such as PPG-3 myristyl ether, trimethylpentanol hydroxyethyl ether; hair fixative polymers, such as amphoteric fixative polymers, cationic fixative polymers, anionic fixative polymers Hair fixative polymers such as amphoteric fixative polymers, cationic fixative polymers, nonionic fixative polymers, silicone graft copolymers; preservatives such as phenoxyethanol, benzoic acid, DMDMH hydantoin, methylisothiazolinone/methylchloroisothiazolinone (MIT/CMIT), benzyl alcohol, methyl paraben, propyl paraben, imidazolidinyl urea; pH regulators such as citric acid, sodium citrate, succinic acid, phosphoric acid, sodium hydroxide, sodium carbonate; salts such as potassium acetate or sodium chloride; colorants; hair oxidizers (bleaching agents) such as hydrogen peroxide, perborates or persulfates; hair reducing agents such as mercaptoacetate; fragrances; and chelating agents such as disodium EDTA.

### EXAMPLES

The present invention is further described below in conjunction with specific embodiments or examples.

Unless otherwise specified, the contents stated are weight percentage. Experimental methods for which specific conditions are not indicated in the Examples are usually performed according to conventional conditions.

Raw Material List

| INCI Name | Manufacturers |
|---|---|
| Piroctone olamine | CLARIANT |
| Cocamidopropyl betaine | CLARIANT |
| Coco-betaine | CLARIANT |
| Sodium lauroyl sarcosinate | CLARIANT |
| Decyl glucoside | BASF |
| Sodium methyl cocoyl taurate | CLARIANT |
| Sodium laureth sulfate | Sasol |
| Cocamide MEA | CLARIANT |
| Polyquaternium-10 | DOW |
| Methylchloroisothiazolinon, | CLARIANT |
| Methylisothiazolinone | |
| Disodium EDTA | Sinopharm Chemical |
| | ReagentCo., Ltd |
| Alcohol | Sinopharm Chemical |
| | ReagentCo., Ltd |
| Propylene glycol | Shanghai Saifu Chemical |
| | Development Co. |
| Butylene glycol | CNOOC Shell Petrochemical |
| | Co. |
| Hexanediol | THOR |
| Pentylene glycol | Ashland |
| Sorbitan caprylate | CLARIANT |

### Test Methods

### Anti-Dandruff Effect Test

Numerous clinical experiments have proven that the reduction of Malassezia on the scalp indicates the reduction of dandruff. Therefore, the anti-dandruff efficacy of the product can be evaluated by the inhibition ability of Malassezia furfur. The anti-dandruff effect of the composition of the present invention was evaluated using the following inhibition zone experimental test method.

Inhibition zone experiment: Inhibition zone is done on modified Leeming-Notman culture medium. Specifically, the concentration of Malassezia is adjusted, 0.3 ml of inoculum was spread over the surface of modified Leeming-Notman culture medium, then incubated 24 h at room temperature. All the samples to be tested are dissolved in sterile solvent. The procedure is mentioned above to check the inhibition zone and the plates which are incubated at 35° C. for 5 days. After incubation, the plates are observed. The inhibition zone is measured using a zone measuring scale and the results are recorded.

Determination of antifungaleffect: the area of inhibition zone ≥300 mm² means extremely effective anti-dandruff effect, i.e. “+++”; 300 mm²> The area of inhibition zone ≥150 mm² means highly effective anti-dandruff effect, i.e. “++”; 100 mm²> The area of inhibition zone ≥50 mm² means medium anti-dandruff effect, i.e. “+”; The area of inhibition zone <50 mm² means no anti-dandruff effect, i.e. “−”

### Preservatives Effect Test

According to European Pharmacopoeia 8th edition, the preservatives effect of the composition is tested by a microbiological test. Criteria for determining the results of the preservative challenge is as follows: 1) Criteria A for bacterial species requires 2 log (or more) reduction on the second day and 3 log (or more) reduction on the 7th day with no increase of bacterial thereafter. No increase in the number of bacteria was detected at the fourteenth and 28th day. Criteria B requires 3 log (or more) reduction on the 14th day with no increase of bacterial thereafter. The number of bacteria does not increase when testing is done on the 28th day. 2). Criterial A for yeast/mould species requires a 2 log reduction at day 14 with no increase thereafter. The B criteria requires a 1 log reduction at day 14 with no increase thereafter.

### Solubility Test

Experimental method: the composition is dissolved in different solvents to determine its dissolution performance. Determination of solubility performance is as follows: solubility >50% means extremely soluble, i.e. “++++”; 50%>solubility≥30% means high solubility, i.e. “+++”; 30%>solubility≥10% means good solubility, i.e. “++”; 10%>solubility≥5% means average solubility, i.e. “+”; solubility <5% means poor solubility, i.e. “−”.

Stability study: The samples of Examples 1-10 and Comparative Examples 1-6 were put into transparent bottles to 80% height (three parallel samples) and placed in a thermostat at room temperature 25° C. and a thermostat at low temperature 4° C., respectively. The samples shall be monitored for one month with observation of their appearance.

Stability determination: clear and homogeneous transparent means stable, i.e. “++++”; precipitation of insoluble matter or appearing uneven means unstable, i.e. “______”.

### Preparation of the Composition

Under normal temperature and pressure, piroctone olamine is added to the alcohol solvent and stirred to dissolve completely. Then the dissolved mixture is added to the surfactant. Finally, the fatty acid ester or oil is added to the mixture and stirred using a mixer to dissolve completely.

The present composition can be added by means of cold process during the formulation, thus saving energy consumption. This is an advantage over processes that require the addition of piroctone olamine to surfactant solution at 70-80° C., or its dispersion in water, or pre-dissolution in polyol.

At room temperature, the composition of the present invention was obtained by mixing and dissolving the alcohol solvent, surfactant, piroctone olamine and fatty acid ester or oil by the ratio as shown in Table 1 according to the preparation method described above. More specifically, Examples 1-10 and Comparative Examples (“CE”) 1-6 are shown in Table 1, in which weight percentage is used for each component. Bacteria inhibition effect and stability evaluation of Examples 1-10 and Comparative Examples 1-6 in Table 1 are shown in Table 5.

TABLE 1
Weight percentage of each component in Examples 1-10 and Comparative Example1-6

| | | | | | | | | | | | CE | CE | CE | CE | CE | CE |
|---|---|---|---|---|---|---|---|---|---|---|---|---|---|---|---|---|
| Ingredient | 1 | 2 | 3 | 4 | 5 | 6 | 7 | 8 | 9 | 10 | 1 | 2 | 3 | 4 | 5 | 6 |
| Piroctone | 8 | 8 | 8 | 8 | 9 | 9.9 | 10 | 10 | 10 | 15 | 4.5 | 5 | 15 | 16 | 18 | 20 |
| olamine | | | | | | | | | | | | | | | | |
| Cocamidopropyl | 10.5 | 3.5 | 12 | 0 | 0 | 10.5 | 10.5 | 10.5 | 20.6 | 0 | 9.5 | 0 | 16.8 | 0 | 0 | 0 |
| betaine | | | | | | | | | | | | | | | | |
| Coco-betaine | 10.5 | 17.5 | 11.2 | 0 | 0 | 0 | 10.5 | 10.5 | 0 | 0 | 0 | 0 | 0 | 0 | 0 | 0 |
| Sodium lauroyl | 0 | 0 | 0 | 10.5 | 21 | 12 | 0 | 0 | 0 | 18.5 | 7 | 0 | 0 | 0 | 0 | 7 |
| sarcosinate | | | | | | | | | | | | | | | | |
| Decyl glucoside | 0 | 1 | 0 | 0 | 0 | 0 | 0 | 0 | 0 | 0 | 0 | 0 | 0 | 0 | 0 | 0 |
| Sodium methyl | 0 | 0 | 1.2 | 0 | 0 | 0 | 0 | 0 | 0 | 0 | 0 | 0 | 0 | 0 | 0 | 0 |
| cocoyl taurate | | | | | | | | | | | | | | | | |
| Sodium laureth | 0 | 0 | 0 | 9 | 0 | 0 | 0 | 0 | 0 | 0 | 9 | 24.3 | 0 | 16.2 | 24 | 15 |
| sulfate | | | | | | | | | | | | | | | | |
| Alcohol | 0 | 0 | 0 | 10 | 0 | 0 | 0 | 0 | 0 | 0 | 0 | 0 | 0 | 0 | 0 | 0 |
| Propylene glycol | 15 | 18 | 9 | 21 | 15 | 20 | 20 | 20 | 10 | 32 | 5 | 10 | 15 | 15 | 0 | 2 |
| Butylene glycol | 0 | 11.5 | 0 | 0 | 10 | 0 | 0 | 9 | 5 | 0 | 5.5 | 1 | 10 | 0 | 2 | 3 |
| Hexanediol | 1.5 | 0 | 0 | 0 | 0 | 0 | 0 | 0 | 0 | 0 | 0 | 0 | 0 | 0 | 0 | 0 |
| Pentylene glycol | 15 | 0 | 12 | 0 | 5 | 5 | 9.5 | 0 | 15 | 0 | 5 | 1 | 10 | 15 | 0 | 3 |
| Sorbitan caprylate | 0.5 | 0.5 | 1 | 1 | 1 | 0.1 | 0.5 | 1 | 1 | 0.5 | 3 | 2 | 2 | 0 | 0 | 2 |
| water | 39 | 40 | 45.6 | 40.5 | 39 | 42.5 | 39 | 39 | 38.4 | 34 | 51.5 | 56.7 | 31.2 | 37.8 | 56 | 48 |

### Preparation of Shampoo Compositions

Shampoos 1-17 were further formulated based on the examples 7-9 and comparative examples 1-2 of the composition according to the present invention, as shown in Table 2 and Table 3, and the shampoos were tested and evaluated for antibacterial effect and preservative effect.

### Shampoo Preparation

Water, sodium laureth sulfate, sodium laureth sarcosinate, sodium methyl cocoyl taurate, decyl glucoside, cocamidopropyl betaine, cocamide MEA, disodium EDTA are put into the emulsifying pot according to the ratios in the Table 2 and Table 3; the mixture is stirred with temperature increasing to 75° C.; polyquaternium-10 is dispersed in water and stirred evenly, with the temperature to 50° C. Such dispersed mixture is put into the aforementioned emulsifying pot, with stirring and lowering the temperature to 45° C. Citric acid and preservative are further added respectively, stirred evenly and dispensed. All products are clear liquids with similar appearance and excellent physical properties compared with typical hair shampoo products available in the market.

Table 2 shows shampoo compositions which were made from Example 6 and Example 7 of the present composition by mixing with the other ingredients as listed with different weight percentages and subsequently tested for anti-dandruff and preservative effects.

TABLE 2

| | Shampoo Examples (weight percentage) | | | | | | |
|---|---|---|---|---|---|---|---|
| | Shampoo | | | | | | |
| Ingredients | 1 | 2 | 3 | 4 | 5 | 6 | 7 |
| Water | 85.94 | 84.92 | 81.86 | 79.185 | 77.16 | 74.01 | 71.91 |
| Disodium EDTA | 0.1 | 0.1 | 0.1 | 0.1 | 0.1 | 0.1 | 0.1 |
| Sodium lauroyl | 0 | 0 | 0 | 7 | 7 | 7 | 7 |
| sarcosinate | | | | | | | |
| Sodium laureth | 9.8 | 9.8 | 9.8 | 0 | 0 | 0 | 0 |
| sulfate | | | | | | | |
| Cocamidopropyl | 1.75 | 1.75 | 1.75 | 5.25 | 5.25 | 5.25 | 5.25 |
| betaine | | | | | | | |
| Sodium methyl | 0 | 0 | 0 | 3 | 3 | 3 | 3 |
| cocoyl taurate | | | | | | | |
| Decyl glucoside | 0 | 0 | 0 | 1 | 1 | 1 | 1 |
| Cocamide MEA | 1 | 1 | 1 | 1 | 1 | 1 | 1 |
| Polyquaternium-10 | 0.3 | 0.3 | 0.3 | 0.3 | 0.3 | 0.3 | 0.3 |
| Methylchloro- | 0.09 | 0.09 | 0.09 | 0.09 | 0.09 | 0.09 | 0.09 |
| isothiazolinon, | | | | | | | |
| Methyliso- | | | | | | | |
| thiazolinone | | | | | | | |
| Citric acid | 0.02 | 0.04 | 0.1 | 0.075 | 0.1 | 0.25 | 0.35 |
| Example 6 | 1 | | | | | | |
| Example 6 | | 2 | | | | | |
| Example 6 | | | 5 | | | | |
| Example 7 | | | | 3 | | | |
| Example 7 | | | | | 5 | | |
| Example 7 | | | | | | 8 | |
| Example 7 | | | | | | | 10 |

Table 3 shows shampoos 8-17 which were formulated by using example 8 of the invented composition and Comparative Examples 1 and 2 with mixing with the other ingredients listed in the Table 3 by different weight percentages, without any additional preservatives, followed by anti-dandruff and preservative effect tests.

TABLE 3

| | Shampoo | | | | | | | | | |
|---|---|---|---|---|---|---|---|---|---|---|
| Ingredients | 8 | 9 | 10 | 11 | 12 | 13 | 14 | 15 | 16 | 17 |
| Water | 87.025 | 86.82 | 86.51 | 86.82 | 86.51 | 86.82 | 86.51 | 85.01 | 81.95 | 76.7 |
| Disodium EDTA | 0.1 | 0.1 | 0.1 | 0.1 | 0.1 | 0.1 | 0.1 | 0.1 | 0.1 | 0.1 |
| Sodium laureth | 9.8 | 9.8 | 9.8 | 9.8 | 9.8 | 9.8 | 9.8 | 9.8 | 9.8 | 9.8 |
| sulfate | | | | | | | | | | |
| Cocamidopropyl | 1.75 | 1.75 | 1.75 | 1.75 | 1.75 | 1.75 | 1.75 | 1.75 | 1.75 | 1.75 |
| betaine | | | | | | | | | | |
| Cocamide MEA | 1 | 1 | 1 | 1 | 1 | 1 | 1 | 1 | 1 | 1 |
| Polyquaternium-10 | 0.3 | 0.3 | 0.3 | 0.3 | 0.3 | 0.3 | 0.3 | 0.3 | 0.3 | 0.3 |
| Citric acid | 0.025 | 0.03 | 0.04 | 0.03 | 0.04 | 0.03 | 0.04 | 0.04 | 0.1 | 0.35 |
| Comparative | | 0.2 | | | | | | | | |
| Example 1 | | | | | | | | | | |
| Comparative | | | 0.5 | | | | | | | |
| Example 1 | | | | | | | | | | |
| Comparative | | | | 0.2 | | | | | | |
| Example 2 | | | | | | | | | | |
| Comparative | 12.975 | 13.18 | 13.49 | 13.18 | 0.5 | | | | | |
| Example 2 | | | | | | | | | | |
| example 8 | | | | | | 0.2 | | | | |
| example 8 | | | | | | | 0.5 | | | |
| example 8 | | | | | | | | 2 | | |
| example 8 | | | | | | | | | 5 | |
| example 8 | | | | | | | | | | 10 |

### Test Results

### Solubility Test Results

Table 4 shows the dissolution properties of examples 1-10 in different solvents.

TABLE 4

| | Solvent | Solubility |
|---|---|---|
| Examples 1-10 | Water | Extremely easy to dissolve |
| | Alcohol | Extremely easy to dissolve |
| | Propylene glycol | Extremely easy to dissolve |
| | Butylene glycol | Extremely easy to dissolve |
| | Pentylene glycol | Extremely easy to dissolve |
| | Hexanediol | Extremely easy to dissolve |

The test results show that the composition of the present invention has excellent solubility. The composition of the present invention can be applied in aqueous products very well. This point can expand the application range of the composition of the present invention, such as tone, spray, serum.

### In Vitro Inhibition Zone Test Results

Table 5 shows the in vitro inhibition against Malasezzia and stability of Examples 1-10 and Comparative Examples 1-6.

TABLE 5
The result of in-vitro bacterial inhibition against Malasezzia
and stability of Examples 1-10 and Comparative Examples 1-6

| | | Low temperature | Room temperature |
|---|---|---|---|
| | Antifungal | stability | stability |
| Example 1 | ++ | +++ | +++ |
| Example 2 | ++ | +++ | +++ |
| Example 3 | ++ | +++ | +++ |
| Example 4 | + | +++ | +++ |
| Example 5 | ++ | +++ | +++ |
| Example 6 | +++ | +++ | +++ |
| Example 7 | +++ | +++ | +++ |
| Example 8 | +++ | +++ | +++ |
| Example 9 | ++ | +++ | +++ |
| Example 10 | +++ | +++ | +++ |
| Comparative | — | − | +++ |
| Example 1 | | | |
| Comparative | — | − | +++ |
| Example 2 | | | |
| Comparative | +++ | − | +++ |
| Example 3 | | | |
| Comparative | ++ | − | − |
| Example 4 | | | |
| Comparative | +++ | − | − |
| Example 5 | | | |
| Comparative | +++ | − | − |
| Example 6 | | | |

Among them, the shampoo compositions were prepared based on the Example 6-8 and Example 10 of the compositions of the present invention, they all have the inhibition zone more than 300 mm², which shows excellent antifungal property, i.e., an extremely effective dandruff removal effect.

Examples 6-7 were applied to form examples of shampoo 1-7. Table 6 below shows in vitro inhibition zone results of shampoo 1-7 made from the present invented composition.

TABLE 6

Shampoo

| | 1 | 2 | 3 | 4 | 5 | 6 | 7 |
|---|---|---|---|---|---|---|---|
| Antifungal | + | ++ | ++ | ++ | +++ | +++ | +++ |

According to the results of the inhibition zone test, the shampoo composition of the present invention has demonstrated excellent inhibition effect, i.e. the composition of the present invention can be used in shampoo formulation with better anti-dandruff effect.

### Preservation Effect Test Results

As stated earlier, shampoo Examples 8-17 were made from Comparative Examples 1-2 and Example 8 of the present composition. The result of preservative challenge are shown in below Table 7.

TABLE 7

| | Shampoo | | | | | | | | | |
|---|---|---|---|---|---|---|---|---|---|---|
| | 8 | 9 | 10 | 11 | 12 | 13 | 14 | 15 | 16 | 17 |
| preservative | Failure | Failure | B | Failure | B | A | A | A | A | A |
| Challenge | | | Standard | | Standard | Standard | Standard | Standard | Standard | Standard |
| Results | | | | | | | | | | |

The test results showed that the preservative challenge (i.e. preservative effect test) in shampoo of the compositions prepared from example 8 of the present invention according to certain weight percentages respectively could reach the standard A, indicating that the compositions of the present invention have excellent preservative effect.

Through the above experiments, it can be seen that the application of the composition of the present invention in the care products makes the care products have both anti-dandruff and preservative effects, and in the preparation process, no high temperature is needed to dissolve and compound the compositions of the present invention, which saves energy.

Various modifications and variations may be made to the present invention without departing from the scope and substance of the invention.

1-15. (canceled)

16. A composition comprising the following components:
A) 0.1-15 wt.-% of piroctone olamine,
B) 17.5-28 wt.-% of surfactant,
C) 15-35 wt.-% of alcohol solvent, and
D) 0.1-2 wt.-% of fatty acid ester or oil,
wherein the weight percentage of each component is based on the total weight of the composition.

17. A composition according to claim 1, wherein the weight percentage of component A is 0.5-15 wt.-%.

18. A composition according to claim 1, wherein the weight percentage of component B is 19-25 wt.-%.

19. A composition according to claim 1, wherein component B is selected from anionic surfactants, amphoteric surfactants, cationic surfactants, nonionic surfactants, and mixtures thereof.

20. A composition according to claim 4, wherein component B is selected from alkyl amidopropyl betaine, alkyl betaine, sodium alkyl acyl sarcosinate, sodium dodecyl polyether sulfate, glucamides, and mixtures thereof.

21. A composition according to claim 1, wherein the weight percentage of component C is 16-30 wt.-%.

22. A composition according to claim 1, wherein component C is selected from propylene glycol, butylene glycol, pentylene glycol, ethanol, hexanediol, and mixtures thereof.

23. A composition according to claim 1, wherein the weight percentage of component D is 0.2-1.5 wt.-%.

24. A composition according to claim 1, wherein component D is selected from sorbitan caprylate, sorbitan oleate, glycerol esters of fatty acids or triglycerides, castor oil, lentil oil, soybean oil, sunflower oil, sorbitan stearate, and mixtures thereof and the composition further comprises E) 25-55 wt.-% of water.

25. A method of preparing a composition according to claim 1 comprising:
a) adding component A to component C and stirring to dissolve completely,
b) adding the above dissolved mixture to component B and mixing further; and
c) adding component D and component E in the mixture and stirring further to dissolve completely.

26. A personal care product or home care product comprising the composition according to claim 1.

27. A personal care product or home care product comprising:
a) 0.2-10 wt.-% of the composition according to claim 1,
b) 8-30 wt.-% of a further surfactant,
c) 0-10 wt.-% of conditioning agent, and
d) water.

28. A personal care product or home care product according to claim 12, wherein the weight percentage of the further surfactant is 10-30 wt.-%.

29. A personal care product or home care product according to claim 12, wherein the further surfactant comprises at least one anionic surfactant, nonionic surfactant, or amphoteric surfactant.

30. A personal care product or home care product according to claim 12, wherein the further surfactant comprises at least one selected from the group consisting of alkyl amidopropyl betaine, alkyl betaine, sodium alkyl acyl sarcosinate, glucamides, and sodium lauryl ether sulfate.
